(19) United States
(12) Patent Application Publication
(10) Pub. No.: US 2006/0106324 A1
(43) Pub. Date: May 18, 2006

(54) Fish biopsy device

(76) Inventors: Rikki C Buckworth, North Territory (AU); Charles R Bryce, Wanguri (AU); Adrian C Donati, Northern Territory (AU)

Correspondence Address: Peter W Peterson;DeLio & Peterson, 121 Whitney Avenue, New Haven, CT, 06510, US

(21) Appl. No.: 10/537,106

(22) PCT Filed: Dec. 2, 2003
(86) PCT No.: PCT/AU03/01609

§ 371 Date: Aug. 22, 2005

(30) Foreign Application Priority Data
Dec. 2, 2002 (AU) 2002953016

(51) Int. Cl. A61B 10/00

(52) U.S. Cl. 600/567

(57) ABSTRACT

The invention relates to a fish biopsy device. The device (1) comprises at least one hook having a tip portion (4), a bend (3) and a shank (2). The tip portion comprises a tube of a non-pliable material having a sharpened end (5) and the tube bore optionally includes a plurality of barbs. The tube has an aperture therein distal the sharpened end; and the bend is formed from a pliable material. The invention also relates to a method of obtaining biopsy material from a fish using the foregoing device.

### TECHNICAL FIELD

This invention relates generally to the area of the monitoring of fishes through analysis of DNA obtained from biopsy material. More particularly, the invention relates to a device which can be used for obtaining biopsy material in situ.

### BACKGROUND ART

Monitoring the impact of fishing is a very real problem in the management of fisheries, attested to by the crisis that exists in the management of fisheries globally, and an extensive scientific literature that examines various aspects of the problem. Tagging is potentially a very powerful tool for monitoring the impact of fisheries. The term “impact “is used in this context to mean estimation of harvest or fishing mortality rates. A great deal of tagging application is for growth and movement studies and while these are important, they don't give an indication of the impact of fishing. Tagging for the estimation of harvest rates is hindered by tag shedding (tags fall out some time after the fish is set free), post-release mortality (the process of capture and subsequent handling to tag fish can be traumatic and can cause cryptic and delayed physiological effects that increase the mortality rate), and unknown reporting rates.

Genetic tagging could be employed for monitoring fisheries. This would involve identifying a set of fish in the population using DNA (this is the “tagging” part of the process) and subsequently screening the catch for matches to the tagged set (this is equivalent to the recaptures in a normal tagging exercise). If the initial tissue sampling does not significantly affect the survival or behaviour of the fish, and the total catch is known, then the three problems referred to above would be overcome. Genetic tagging has been used to examine population sizes and relatedness for several terrestrial species such as wolves, bears and wombats, and marine mammals. An advantage is that very little tissue is needed and non-invasive approaches are particularly attractive for dangerous or conservation-important species (use of hairs from rubbing posts, collection of scats and the like). In the case of marine mammals, special harpoons and darts have been developed to collect the tissue sample.

An additional problem with tagging is that it is expensive: capture of the fish in the first place is a major component of the expense of tagging programs and often precludes its application as a monitoring tool (to determine the harvest rate, tagging is typically one-off experimentation rather than an annual or more frequent event that monitoring requires).

It would therefore be desirable to have available a device for gathering small samples of tissue from fish remotely so that fish do not have to be captured for that purpose. The tissue would be used for molecular genetic typing (for example, microsatellite or mitochondrial DNA sequencing) for application in genetic mark recapture for estimation of mortality and movement rates of fish, and for studies of fish stock structure (such as spatial and temporal patterns in the genetics of the species being examined). These small amounts of tissue might also be used for epidemiological studies (spatial/temporal patterns in diseases), chemical testing for toxicology studies (for example, concentrations of heavy metals), or for other physiological measures.

### SUMMARY OF THE INVENTION

The object of the invention is to provide a device which can be used for obtaining biopsy material from a fish without having to physically catch the fish.

In a first embodiment, the invention provides a fish biopsy device comprising at least one hook having a tip portion, a bend, and a shank, wherein:

said tip portion comprises a tube of a non-pliable material having a sharpened end, wherein said tube bore optionally includes a plurality of barbs, and wherein said tube has an aperture therein distal said sharpened end; and

said bend is formed from a pliable material.

In a second embodiment, the invention provides a method of obtaining biopsy material from a fish, said method comprising the steps of:
i) providing a line with a device according to the first embodiment attached thereto;
ii) allowing a fish to strike said device;
iii) recovering said device after release from said fish; and
iv) obtaining said biopsy material from the tip portion of the device.

With regard to the device according to the first embodiment, those of skill in the art will appreciate that it in part functions like a conventional fish hook and in this regard it is used together with a fishing line. For attachment of a line, devices advantageously but optionally include an eye, typically at the end of the shank portion of a device as with conventional fish hooks. The device is typically used in conjunction with a lure to attract fish thereto. However, use of a lure is not essential and devices can even be used with other attractants or bait attached thereto.

The principal of operation of a device according to the invention is as follows: when a fish attacks the device (normally on being attracted thereto by a lure), the force of the attack and movement of the device via the line to which it is attached brings the tip portion into contact with the flesh on the inside of the mouth of the fish. The sharpened end of the tip either penetrates or scrapes along the flesh and thereby a tissue sample is driven into the bore of the tip portion where it is retained by the barbs if present. In embodiments of the device that do not have barbs in the tip portion, the tissue sample is held therein by being compressed when driven into the portion.

After the initial attack on the device, the actions of the fish and/or the continued movement of the line cause a plastic deformation of the pliable bend. As a consequence the bend straightens and this, coupled with the fact that the tip portion is not externally barbed, disengages the device and frees the fish. This deformation of the hook also inactivates the device preventing sampling from a second fish.

With further reference to a device according to the first embodiment, the tubular tip portion is typically fabricated from a metals material such as steel. A particularly preferred material is stainless steel. However, the tip portion can be fabricated from any suitable material such as a plastic, glass or ceramic material meeting the following requirements:
it will not be deformed when in use (that is, it is sufficiently hard); and
it can be machined to provide an end with sufficient sharpness for penetrating flesh on the inside or outside of the mouth of a fish.
   The same requirements apply to tip portions of a metals material.

The sharpened end of the tip portion can be provided by chamfering the edges of the tube forming the portion or by castellating the tip. Typically, however, the sharpened end is provided by beveling the end at an angle of 25 to 45° to the axis of the tube. A preferred bevel angle is 30° to the axis of the tube which provides a sharpened end like that of a hypodermic syringe. With tip portions comprising beveled ends, the portion is preferably positioned so that the point formed by the bevel is on the outer side of the hook.

Ends of tip portions can be sharpened using any of the techniques known to those of skill in the art including, grinding, cutting, or chemical or laser sharpening.

The plurality of barbs can be machined into the inside walls of the tube forming the tip portion. Alternatively, the barbs can be on a member or member projecting into the bore of the tip portion from the end that adjoins the other sections of the hook. This will be explained in greater detail below.

In embodiments where the tip portion does not include internal barbs, the sharpened end advantageously has an internal diameter that is no greater than the bore diameter of the entire tip portion. Preferably however, the sharpened end has an internal diameter that is slightly less than that of the bore of the tip portion. In the preferment, the sharpened end provides a constriction which helps retain the tissue sample within the tip portion. Devices with the forgoing type of tip portion are particularly suited for obtaining biopsy material from fish with small mouths such as luderick and whiting.

The aperture in the tip portion of the hook is merely to allow for pressure equalization when tissue is driven into the portion through the sharpened end. The aperture can take any form and is typically a hole or slot cut through the wall of the tube forming the portion.

The bend portion of the hook of a device is formed from a material that:
has sufficient rigidity so that the sharpened end of the tip portion contacts the flesh of the fish at the time of an initial strike on the device; but
is sufficiently pliable to straighten under the action of the fish and/or force applied via the line attached to the device to disengage the tip portion and free the fish.

Those of skill in the art will appreciate the types of material which can be used for fabricating the bend and that these materials include plastics, ceramics, metals and alloys. A preferred material is copper which can be in the form of a tube or a solid wire. Other suitable materials include aluminium alloys. The section of material forming the bend can be provided as a solid—that is, as a section of wire—or in tubular form.

A device can include a plurality of hooks. The plurality of hooks can be linked via a common shank portion or by merely fixing a plurality of shanks together. The hooks of a device comprising a plurality of hooks can be positioned longitudinally with respect to each other, laterally with respect to each other, or a combination of the foregoing possibilities. Further details of devices with multiple hooks will be given below.

The material used for the bend of a device can also be used for the shank portion. That is, the bend and shank can be formed from a single piece of material.

As indicated above, a preferred material for the bend portion of a device is copper which is advantageously in the form of a tube. Alternatively, the bend can be formed from annealed copper wire as can the shaft of a device.

The tip portion is typically joined to the bend by crimping. However, any suitable method can be used to fix the tip portion to the bend. Advantageously, the tip portion can be tapped for screw attachment to the bend. This is particularly the case for a tip portion fabricated from a metal such as stainless steel. Plastics, carbon, ceramic or glass tips are advantageously chemically bonded (glued) to the bend.

The plurality of barbs are conveniently provided in the form of dental broaches which are minute file-like articles. The ends of broaches distal the point of the tip portion can be fixed into the end of the bend to which the tip portion is secured. Alternatively, the ends of broaches can be fixed into a plug of material in the bore of the tip portion. Tip portions with broaches fixed thereinto which are also threaded for attachment to the bend would allow reuse of the tips.

The plurality of internal barbs can also be provided by including small gauge fish hooks within the tip portion, straightened and cut to an appropriate length fit within the portion. Any metal rod of suitable diameter cut to length and with sharp-edged grooves, teeth or threads cut thereon can also be used to provide the internal barbing.

Devices can be configured for use with different species of fish or for use in different fisheries. Lures can also be positioned as appropriate for the fish from which biopsies are to be collected may require different conformations of positioning of the device on the lure body. Devices can thus be adapted to different behaviours of fish arising from different species, fish size, environmental conditions, deployment conditions or lure bodies. Additionally, devices can be sized as appropriate for the target fish and the distance between the tip portion and shank can also be varied. With regard to devices comprising more than one hook, there will be a higher probability of a fish or fishes successfully coming into contact with a tip portion (yielding tissue) when the device is struck and the possibility of a double strike on device producing distinct samples from more than one fish, each in separate hooks on the same multi-hook device.

As indicated above, devices can be used with bait rather than lures or the like. The bait is advantageously attached to the bend portion of the device. The bait is necessarily from a different species, so that the DNA assessment indicates the target species. Some difference in shape is advantageous with devices used with bait such that the tip portion of the device is bent inward to face the shank. This will usually ensure that that the fish takes the device fully into its mouth. A device of this type will be described in greater detail below.

Devices according to the invention are suitable for taking biopsies from species including those referred to in descriptions below of particular devices. Devices are also suitable for use with species of tunas and similar species (Family Scombridae) using longlines or drop lines (vertical long-lines). The principal species/fisheries in this group are the snappers and emperors (Families Lutjanidae and Lethrinidae) and similar species such as breams (porgies, Family Sparidae), for example pink snapper, goldband snapper, golden snapper, emperors and groupers (Family Serranidae, often called “cods” in Australia) such as coral trout, croakers (Family Sciaenidae, that includes mulloway, jewfishes, terraglin) and gemfish and related species, and even luderick and drummer (Girellidae). Devices comprising very fine shanks and tips can also be used in squid/calamary fisheries.

Having broadly described the invention specific devices will now be exemplified with reference to the accompanying drawings briefly described hereafter.

### BRIEF DESCRIPTION OF THE DRAWINGS

FIG. 1 is an elevational view of a device according to the invention.

FIG. 2 is an amplification of the tip portion of the device of FIG. 1 with certain components in phantom.

FIG. 3 is an elevational view of the device of FIG. 1 after utilisation for obtaining a fish biopsy.

FIG. 4 is an elevational view of a device designed for obtaining biopsies from snappers, or pelagic fishes.

FIGS. 5 to 9 are elevational views of alternative devices according to the invention.

### BEST MODE AND OTHER MODES OF CARRYING OUT THE INVENTION

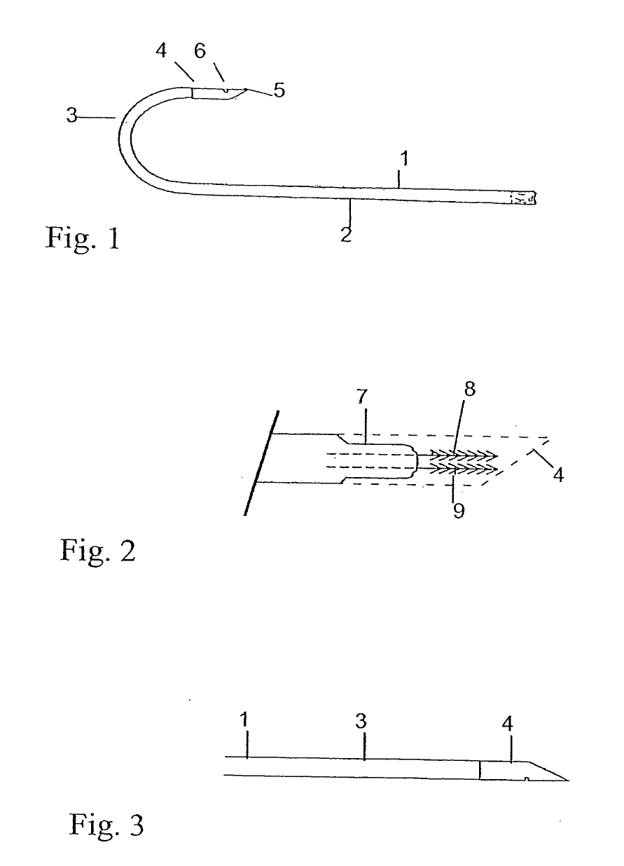

Referring firstly to FIG. 1, there is shown device 1 comprising a hook having a shank 2, a bend 3 and a tip portion 4. Tip portion 4 has a beveled end 5 and an aperture 6 therein in the form of a slot. Tip portion 4 is formed from stainless steel tubing of 2.8 mm OD and has an overall length of 15 mm. The bevel at end 5 is at 30° to the axis of the tube forming the tip portion. Bend 3 and shank 2 are formed from 2.8 mm OD copper tube. The shank and tip portions are about 22 mm apart. Shank 2 has at the end distal bend 3 an eye, not shown in the drawing, for attaching a line in conjunction with a lure if desired.

In FIG. 2, portion of device 1 comprising tip portion 4 is shown in greater detail. Tip portion 4 is shown in phantom whereby it can be seen that it is crimped over the end 7 of the copper tube forming bend 3. End 7 of bend 3 is in fact reduced in diameter to allow tip portion 4 to be fitted thereover. Two coarse dental broaches 8 and 9 of 20 mm length extend from end 7 and lie within the bore of the tube forming tip portion 4. These broaches provide the plurality of internal barbs for retaining tissue within the bore of tip portion 4.

It can be appreciated from FIGS. 1 and 2 that when a fish strikes device 1, tip portion 4 will contact the cheek or mouth of the fish. Bevel 5 will scrape tissue from the flesh forming the cheek or mouth which tissue will lodge in the bore of tip portion 4. Due to the pliable nature of bend 3, the action of the fish after striking device 1 and/or force applied to the device via the line attached thereto causes the bend to straighten. As a result, device 1 adopts the configuration shown in FIG. 3 by which time the device will have been released from the mouth of the fish. It will be further appreciated that when in the FIG. 3 configuration, device 1 cannot collect tissue and has thus been inactivated. A device in the FIG. 1 configuration can therefore collect only a single sample which is crucial for accurate genetic analysis.

The device of FIGS. 1 and 2 is particularly suited for obtaining tissue samples from fishes including those found in the following fisheries: Spanish mackerel fisheries such as those of the Australian states of Queensland, and Western Australia, the Northern Territory of Australia, and the Torres Strait fisheries for narrow-barred Spanish mackerel (Scomberomorus commerson), grey mackerel (S. semifasciatus), spotted mackerel (S. munroi), Queensland school mackerel (S. queenslandicus) and Atlantic/Gulf of Mexico fisheries for King mackerel (S. cavalla) and Spanish mackerel (S. maculatus), and fisheries for other Scomberomorus species; marlin/billfish fisheries; tuna fisheries such as those in Australia for southern bluefin tuna, yellowfin, skipjack and albacore tuna, as well as minor fisheries for the longtail tuna and other small tunas and tuna-like species; swordfish; yellowtail kingfish and other carangid species (trevallies and jacks); large predatory reef fishes such as coral trouts and other groupers (Family Serranidae), barramundi and other perch and perch like fishes (including Murray cod, Maquarie perch, jungle perch and golden perch), Australian bass; salmonids (salmons and trouts); estuarine and coastal fishes such as breams (Pagridae) and flatheads (Platycephalidae) and, shark fisheries.

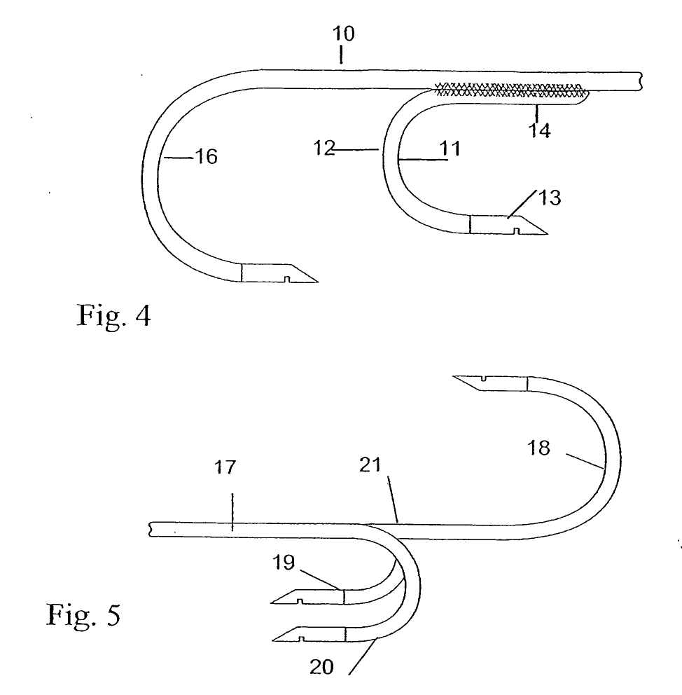

Alternative devices were shown in FIGS. 4 to 8. These alternative devices are all designed for use with pelagic predatory fishes, such as Spanish mackerels (Scomberomorus spp.) or tunas (Thunnus spp.) and trevallies and jacks (Family Carangidae). The FIG. 4 device 10 is similar to the FIGS. 1 and 2 device but includes a second hook 11 comprising a bend 12 fabricated from copper tube and a stainless steel tip portion 13. Shank 14 of second hook 11 is welded to shank 15 of first hook 16 so that hook 11 lies in essentially the same plane as hook 16. Device 10 is designed for attachment to a lure or for deployment with bait or other attractants.

Device 17 of FIG. 5 has a first hook 18 like that of the FIGS. 1 and 2 device but has two further hooks 19 and 20 fixed to shank 21 of the first hook upwardly of the hook and on the opposite side of the device.

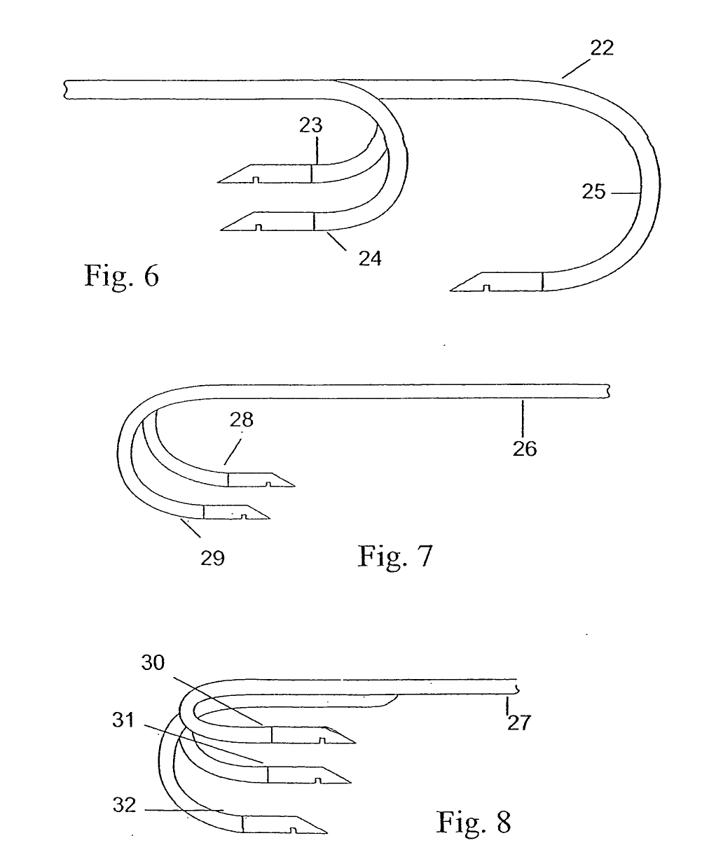

FIG. 6 depicts a device 22 that is in essence a claw and is like the device of FIG. 5 save that second and third hooks 23 and 24, respectively, are on the same side of the device as first hook 25.

The devices depicted in FIGS. 7 and 8, items 26 and 27 respectively, are similar in that they comprise a plurality of hooks radially disposed about an axis with the points of tip portions lying in essentially the same plane. Device 26 has two hooks 28 and 29 while device 27 has three hooks 30 to 32. The hooks of device 26 are radially separated by an angle of about 90° while the outer hooks of device 27 are radially separated from the middle hook by an angle of about 45°.

Other details of the FIGS. 4 to 8 devices not specified above are the same as the FIGS. 1 and 2 device.

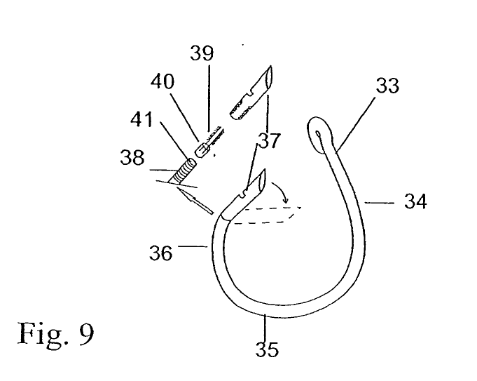

The device shown in FIG. 9 is designed specifically for use with long-lines or drop-lines and thus is targeted at species for which these methods are used. Device 33 is similar to a ‘tuna circle’ hook in shape, in that shaft 34 and bend 35 are contiguous forming an approximate circle, distal end 36 bending over so that tip portion 37 when attached faces toward shaft 34. The exact angle is varied depending upon target species and deployment.

The exploded portion of FIG. 9 shows the screw method for attachment of tip portion 37. Threaded distal end 38 of bend 35, tip portion 37, and two dental broaches (one of which is item 39) held in a plug of material 40 can be seen in the exploded portion. The plug of material 40 is cemented into the bore 41 of threaded end 38 of bend 35. Bait or any suitable material for attracting fish (bright-coloured or shiny material such as used for fishing flies) is attached to the bend of device 33 for use in obtaining tissue samples.

It will be appreciated that many changes can be made to the devices exemplified above without departing from the broad ambit and scope of the invention.

The terms “comprise” and variants of the term such as “comprises” or “comprising” are used herein to denote the inclusion of a stated integer or stated integers but not to exclude any other integer or any other integers, unless in the context or usage an exclusive interpretation of the term is required.

1. A fish biopsy device comprising at least one hook having a tip portion, a bend, and a shank, wherein:
said tip portion comprises a tube of a non-pliable material having a sharpened end, wherein said tube bore optionally includes a plurality of barbs, and wherein said tube has an aperture therein distal said sharpened end; and
said bend is formed from a pliable material.

2. The device of claim 1, wherein said tubular tip portion is fabricated from a material selected from the group consisting of metals, plastics, glass and ceramics.

3. The device of claim 2, wherein said tubular tip portion is fabricated from stainless steel.

4. The device of claim 1, wherein said sharpened end of the tip portion is provided by chamfering the edges of the tube forming the portion or by castellating the tip.

5. The device of claim 1, wherein said sharpened end of the tip portion is a bevel having an angle of 25 to 45° to the axis of the tube forming the portion.

6. The device of claim 5, wherein said bevel has an angle of 30° to the axis of the tube forming the portion.

7. The device of claim 5, wherein said tip portion is positioned so that the point formed by the bevel is on the outer side of the hook.

8. The device of claim 1, wherein said tip portion includes barbs which are machined into the inside walls of the tube forming the tip portion.

9. The device of claim 1, wherein said tip portion includes barbs comprising at least one member projecting into the bore of the tip portion from the end that adjoins the bend of the hook.

10. The device of claim 9, wherein said at least one member comprises a dental broach.

11. The device of claim 1, wherein said tip portion does not include internal barbs and the sharpened end has an internal diameter that is slightly less than the bore of the tip portion.

12. The device of claim 1, wherein said aperture is a hole or slot cut through the wall of the tube forming the tip portion.

13. The device of claim 1, wherein said bend is fabricated from a material selected from the group consisting of plastics, ceramics, metals and alloys.

14. The device of claim 13, wherein said bend is fabricated from copper.

15. The device of claim 13, wherein said bend is fabricated from aluminium alloy.

16. The device of claim 13, wherein said bend is in the form of a tube or a solid wire.

17. The device of claim 1, wherein said bend and shank are formed from a single piece of material.

18. The device of claim 1, comprising a plurality of hooks.

19. The device of claim 18, wherein said plurality of hooks are linked by a common shank.

20. The device of claim 18, wherein each of said plurality of hooks has a shank and the shanks are linked to form the device.

21. The device of claim 18, wherein said hooks are positioned longitudinally with respect to each other, laterally with respect to each other, or a combination of longitudinally and laterally.

22. The device of claim 18, comprising two hooks.

23. The device of claim 18, comprising three hooks.

24. The device of claim 1, wherein said tip portion is threadingly engaged by said bend.

25. The device of claim 1, wherein said shank has an eye at the end thereof for attachment of a line to said device.

26. A method of obtaining biopsy material from a fish, said method comprising the steps of:
i) providing a line with a device according to any one of claims 1 to 25 attached thereto;
ii) allowing a fish to strike said device;
iii) recovering said device after release from said fish; and
iv) obtaining said biopsy material from the tip portion of the device.

27. The method of claim 26, wherein said hook has bait attached thereto, wherein said bait is from a species different to said target species.

28. The method of claim 26, wherein said hook is used in conjunction with a lure.
